(19) United States
(12) Patent Application Publication
(10) Pub. No.: US 2023/0256123 A1
(43) Pub. Date: Aug. 17, 2023

(54) STORAGE COMPARTMENT FOR A VEHICLE

(71) Applicant: MERCEDES-BENZ GROUP AG, Stuttgart (DE)

(72) Inventors: Valentin MAIER, Ispringen (DE); Benno STAMP, Stuttgart (DE); Christoph MENZEL, Stuttgart (DE)

(21) Appl. No.: 18/014,520

(22) PCT Filed: Jun. 28, 2021
(86) PCT No.: PCT/EP2021/067688

§ 371 Date: Jan. 5, 2023

(30) Foreign Application Priority Data
Jul. 6, 2020 (DE) 10 2020 004 058.6

(51) Int. Cl. A61L 2/10; B60R 7/04; A61L 2/26

(52) U.S. Cl. CPC A61L 2/10; B60R 7/04; A61L 2/26; A61L 2202/11

(57) ABSTRACT

A vehicle storage compartment includes an integrated disinfection device with at least one light source that emits UV-C radiation. A support for supporting and disinfecting an item has a surface structure that reflects the UV-C radiation from the at least one light source so that the reflected UV-C radiation irradiates a side of the item facing the support.

### BACKGROUND AND SUMMARY OF THE INVENTION

Exemplary embodiments of the invention relate to a storage compartment for a vehicle with an integrated disinfection device with at least one light source that emits UV-C radiation.

A storage compartment for a vehicle is known from KR 10 1517 694 B1, wherein the storage compartment has a wireless charging device and LED modules on two sides of the housing. Furthermore, the storage compartment is designed to be displaceable in the X direction. The LED modules are designed in such a way that an item stored in the support can be disinfected.

Exemplary embodiments of the invention are directed to a storage compartment for a vehicle with an integrated disinfection device.

A storage compartment for a vehicle comprises an integrated disinfection device with at least one light source that emits UV-C radiation. According to the invention a support for supporting and disinfecting an item has a surface structure that reflects the UV-C radiation from the at least one light source, which is designed so that the reflected UV-C radiation irradiates a side of the item facing the support. Here, the surface structure is designed in such a way that the side of the stored item facing the support is spaced apart from a deepest point of the surface structure.

It is possible to irradiate the side of the item facing the support, when the item is stored, with the UV-C radiation, by means of the surface structure of the support without there being any need to rotate the item. The side facing the support is thus not in shadow and it is therefore not necessary to increase the number of UV-C radiation-emitting light sources.

It is thereby possible for the UV-C radiation to be reflected and reach the side of the item facing the support. A beam path of the reflected UV-C radiation can therefore be created so that the reflected UV-C radiation at least partially irradiates the side of the item facing the support.

In a further embodiment, the surface structure of the support is designed so that at least a part of the reflected UV-C radiation is diffused in relation to the side of the stored item facing the support. In particular, the reflected UV-C radiation is diffused in such a way that the whole of the side of the item facing the support is reached and thus disinfected.

Furthermore, a development of the storage compartment provides that the surface structure is designed as a wave shape. The wave shape has both support regions as well as lower regions, so that the UV-C radiation is efficiently reflected in order to irradiate the side of the item facing the support.

In order to enable the item to be supported and still have its side facing the support reached by the reflected UV-C radiation, peaks of the wave shape are formed with varying heights. Here, the highest peaks form the support regions for the item, wherein a number of the support regions is chosen in such a way that the item is supported securely and shaking, as well as noise caused by this, can be eliminated as far as possible.

In a possible development, the support comprises a reflecting support structure, which has the surface structure, in particular formed as the wave shape.

In an alternative embodiment, the support comprises a support structure on which a reflective layer is arranged, by means of which the UV-C radiation emitted by means of the light source is reflected, so that it is diffused and the reflected UV-C radiation at least partially irradiates the side of the item facing the support. Here, the reflective layer can comprise silver and/or gold.

Additionally, a possible further embodiment provides that a protective layer that transmits UV-C radiation is arranged on the reflective layer, by means of which the reflective layer can be protected from damage, for example scratches. Furthermore, the protective layer protects the reflective layer against other external influences and against moisture.

An embodiment in particular provides that the protective layer comprises silicon dioxide, since this transmits the UV-C radiation as much as possible, so the reflected UV-C radiation reaches the underside of the item.

### BRIEF DESCRIPTION OF THE DRAWING FIGURES

Exemplary embodiments of the invention are described in more detail by reference to the drawings in the following.

Here:

FIG. 1 schematically shows a storage compartment with light sources of a disinfection device arranged under a support,

FIG. 2 schematically shows a support with a surface structure and a light source of the disinfection device arranged above the support,

FIG. 3 schematically shows a perspective view of the support with the surface structure and

FIG. 4 schematically shows a construction of the support.

Components that correspond to each other are provided with the same reference numerals in all figures.

### DETAILED DESCRIPTION

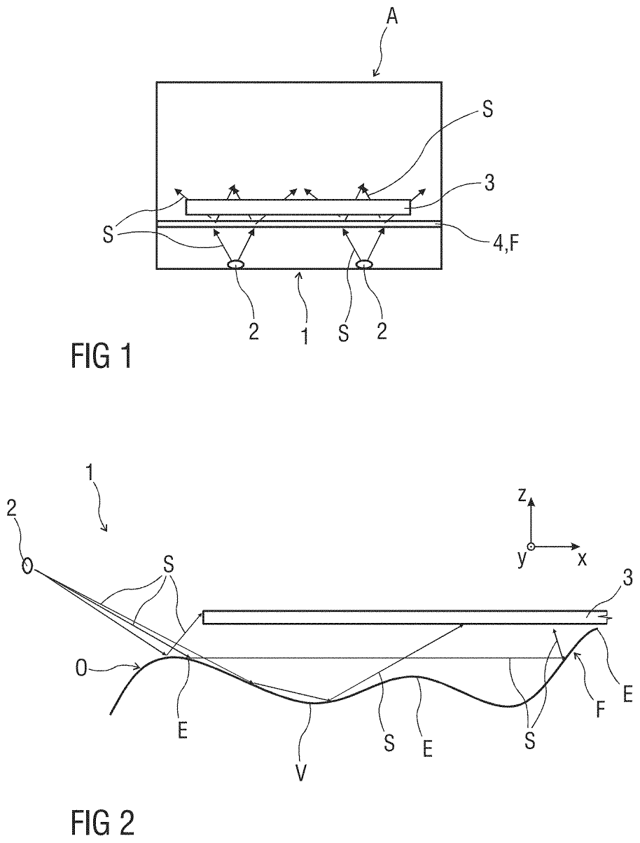

FIG. 1 shows, as an example, a storage compartment A for a vehicle, wherein a disinfection device 1 is integrated into the storage compartment, which, according to FIG. 1, comprises two UV-C radiation S emitting light sources 2.

The storage compartment A serves for storing an item 3, for example for a smartphone, which is disinfected by means of the UV-C radiation S while the item is stored.

Typically, irradiating a side of the item 3 facing the support presents a technical problem since the side facing the support is in shadow when UV-C radiation S is emitted onto the item 3 from above. According to the present exemplary embodiment, the side of the item 3 facing the support is an underside of this and is referred to as the underside in the following.

According to FIG. 1, the technical problem of irradiating the underside of the item 3 is solved in that a medium 4 that transmits UV-C radiation S, which is in particular made of a quartz glass sheet, forms a support F of the item 3.

The light sources 2 are arranged between a floor of the storage compartment A and the medium 4 that forms the support F, by means of which the emitted UV-C radiation S is scattered so that this is diffused.

Because the light sources 2 are arranged under the item 3 to be irradiated, the underside is irradiated, wherein an increased space requirement exists for the arrangement of the light sources 2 in this way. Space requirements for integrating the disinfection device 1 into the storage compartment A are therefore increased, whereby higher costs as well as disadvantages related to installation space-dictated construction options and complexity arise.

In order to be able to substantially completely irradiate the item 3 with the UV-C radiation while it is stored in the storage compartment A and thus to be able to disinfect it, without arranging at least one light source 2 that emits UV-C radiation S under the support F, the storage compartment A is designed with the integrated disinfection device 1, as described in the following.

FIG. 2 shows a support F with a surface structure O, on which the item 3 is placed.

The surface structure O comprises peaks E and indentations V here so that the surface structure O is formed in a wave shape. Here, the highest peaks E form support regions on which the item 3 lies, whereas the item 3 is spaced apart from the indentations V, in particular from their deepest points, while it is stored. In addition, the support F is designed to be reflective.

Alternatively, or additionally, the surface structure O of the support F can also have another suitable shape.

The light source 2 that emits UV-C radiation S, which is in particular a light-emitting diode, is arranged in a corner region or on a side wall of the storage compartment A. The UV-C radiation S that therefore comes from above and from the side is reflected by the reflecting surface structure O in such a way that the UV-C radiation S is directed onto the underside of the item 3 and reaches this.

The surface structure O is additionally designed in such a way that the UV-C radiation S is not reflected directly perpendicularly upwards when it strikes the support F, but rather a part of the reflected UV-C radiation S is diffused at a comparatively very shallow angle in one plane. It is thus possible that the underside of the item 3 is almost completely irradiated by the reflected UV-C radiation S.

A diffusion of the reflected UV-C radiation S is achieved by means of the wave-shaped surface structure O and/or by means of another suitable surface structure O, which has support regions, i.e., peaks E and indentations V.

The UV-C radiation S emitted from light sources 2 in the form of the light-emitting diode has a plurality of different radiation angles, wherein a radiation cone of a light-emitting diode has a range of, for example, 120°.

Therefore, a plurality of input and output angles arise from the wave-shaped surface structure O of the support F, depending on where the UV-C radiation S hits the surface structure O. Such a point of the surface structure O where the UV-C radiation S is reflected is referred to as the reflection point R, wherein the surface structure O can be referred to as the reflection geometry.

Due to the different heights of the peaks E, some UV-C rays S are reflected directly upwards in the direction of a vertical axis z, whereas other UV-C rays S are diffused in a horizontal direction x and, elsewhere on the surface structure O, are reflected in the direction of the vertical axis z.

Peaks E with different heights that are offset three-dimensionally, e.g., in the lateral direction y, also contribute to the UV-C rays S being diffused across a comparatively large surface. These so-called high points also have to be formed in the lateral direction y so that the UV-C radiation S that hits them from the side undergoes such an effect.

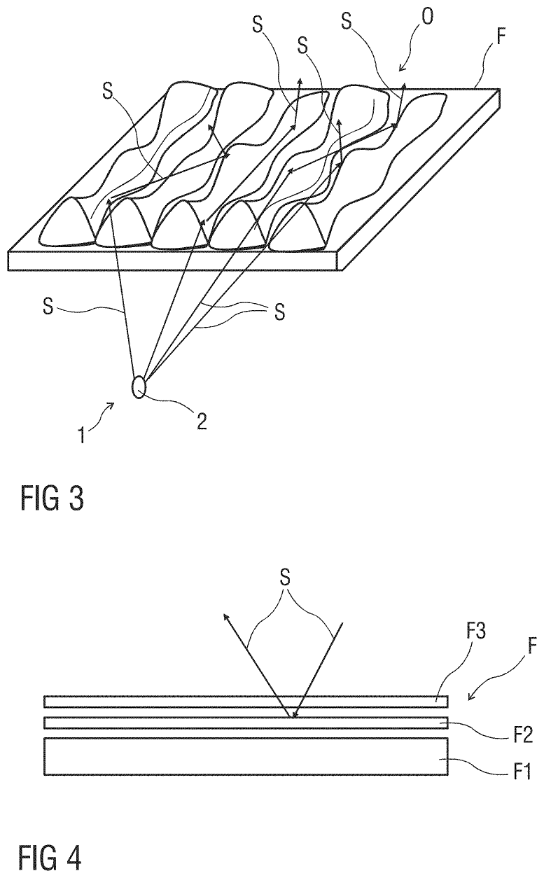

The exemplary embodiment shown in FIG. 2 illustrates a cutout of the support F and thus of the surface structure O, wherein a perspective view of the support F with the surface structure O is shown in FIG. 3.

Due to the shadow of the stored item 3, the reflected UV-C rays S only reach a part of the surface structure O, wherein the reflected UV-C rays S nevertheless spread out in the horizontal direction x and almost completely irradiate the underside of the item 3.

According to the exemplary embodiment shown in FIG. 2, further support regions of the surface structure O, so of the support F, can be located in the lateral direction y.

As is shown in FIG. 3, the UV-C rays S only reach a certain region of the reflection geometry, so of the wave-shaped surface structure O, due to the shadow of the item 3, wherein the UV-C rays S nevertheless spread out in the horizontal direction x and can thus almost completely irradiate the underside of the item 3.

FIG. 4 shows an exemplary construction of the support F without the molded surface structure O.

The support F as support structure F1 can, for example, be formed from a comparatively flexible thermoplastic elastomer, wherein less flexible plastics, such as e.g., polypropylene, polycarbonate and/or acrylonitrile butadiene styrene, can also be used.

An embodiment made of, for example, polished aluminum, stainless steel and/or another reflective metal is also conceivable.

If the support structure F1 of the support F is made from a non-reflective plastic and/or another non-reflective material, it is necessary to apply a reflective layer F2 to the support structure F1, so that UV-C rays S that hit this are reflected, in order to irradiate the underside of the stored item 3.

For example, an aluminum layer, a gold layer and/or a silver layer can be applied to the support structure F1 as a reflective layer F2, e.g., by means of a vacuum-based coating process or by means of thin-layer technology.

A protective layer F3 is arranged on the reflective layer F2 to protect the reflective layer F2 against external influences and/or moisture, the protective layer being transmissive at least regarding the UV-C radiation S. Since quartz glass has this property, the protective layer F3 can comprise silicon dioxide.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-7. (canceled)

8. A vehicle storage compartment, comprising:
a disinfection device integrated into the vehicle storage compartment and including at least one light source configured to emit UV-C radiation; and
a support configured to support and disinfect an item in the vehicle storage compartment, the support has a surface structure configured to reflect the UV-C radiation from the at least one light source in such a way that the reflected UV-C radiation irradiates a side of the item facing the support,
wherein the side of the stored item facing the support is spaced apart from a deepest point of the surface structure, and
wherein the support comprises a support structure, on which a reflective layer is arranged.

9. The vehicle storage compartment of claim 8, wherein the surface structure is configured so that at least a part of the reflected UV-C radiation is diffused in relation to the side of the stored item facing the support.

10. The vehicle storage compartment of claim 8, wherein the surface structure is wave shaped.

11. The vehicle storage compartment of claim 10, wherein peaks of the wave shaped surface structure have varying heights.

12. The vehicle storage compartment of claim 8, wherein the support comprises a reflective support structure.

13. The vehicle storage compartment of claim 8, further comprising:
a protective layer, which at least transmits UV-C radiation, is arranged on the reflective layer.

14. The vehicle storage compartment according to claim 13, wherein the protective layer comprises silicon dioxide.
